(19) United States
(12) Patent Application Publication
(10) Pub. No.: US 2011/0021933 A1
(43) Pub. Date: Jan. 27, 2011

(54) Methods and Systems for Use in Determination of a Patient's Heart Condition

(75) Inventors: Naira Radzievsky, Haifa (IL); Surik Papyan, Haifa (IL)

Correspondence Address: HOUSTON ELISEEVA, 420 BEDFORD ST, SUITE 155, LEXINGTON, MA, 02420, US

(73) Assignee: DeepBreeze Ltd., Or Akiva, IL (US)

(21) Appl. No.: 12/933,425

(22) PCT Filed: Mar. 29, 2009
(86) PCT No.: PCT/IL09/00326

§ 371 Date: Sep. 19, 2010

Related U.S. Application Data
(60) Provisional application No. 61/064,739, filed on Mar. 24, 2008.

(51) Int. Cl. A61B 5/0452; A61B 7/04

(52) U.S. Cl. 600/513; 600/509

(57) ABSTRACT

A method and system are presented for use in monitoring a subject's heart conditions such as ejection fraction and cardiac synchrony. For determination of the ejection fraction, ECG measured data is processed. This ECG measured data comprises a first measured data portion indicative of a first time profile of an electrical signal measured on a planar pair of electrodes applied on a patient during a measurement session and a second measured data portion indicative a second time profile of an electrical signal measured on a diagonal pair of electrodes applied to said patient during said measurement session. The processing comprises comparing the first and second data portions to identify a time shift between a predetermined event in the first and second time profiles. For the determination of heart synchrony, first and second measured data portions corresponding to respectively an ECG measurement and an acoustic measurement both obtained from a patient during the same measurement session of a duration including at least two cardiovascular cycles, are processed. Such processing comprises identifying predetermined first and second events in each of said at least two cycles in the first and second measured data portions, respectively, determining a relation between the first and second events for each of the at least two cycles, determining a degree of fluctuation of said relation in the at least two cycles.

### FIELD OF THE INVENTION

This invention is generally in the field of medical devices, and relates to methods and systems for use in the determination of a patient's heart condition.

### BACKGROUND

Monitoring of the heart condition is important for timely detection of various abnormalities such as cardiac arrhythmias, conduction abnormalities, ventricular hypertrophy, myocardial infarction, electrolyte derangements, etc.

Assessment of left ventricular (LV) function plays a central role in diagnosis and treatment of heart disease. Cardiac resynchronization therapy (CRT) improves quality of life, reduces hospitalizations due to heart failure, and reduces mortality

The heart condition is generally characterized by various measurable parameters, including among others an ejection fraction and a cardiac synchrony. The ejection fraction can directly be calculated using echocardiology, cardiac MRI, CT, etc. On the other hand, an effect of reduction in the ejection fraction can be detected via ECG-QRS based measurements: increase in the QRS is indicative of a reduction of the ejection fraction. As for the cardiac synchrony, it is typically detected by echocardiography.

### GENERAL DESCRIPTION

The present invention provides novel methods and systems for monitoring a patient's heart conditions, such as ejection fraction and cardiac synchrony conditions.

According to one broad aspect of the invention, a monitoring method comprises extracting information about the subject's ejection fraction from ECG-based measured data. The inventors have found that a relation between a measurement taken from a diagonal pair of electrodes and a measurement taken from a planar pair of electrodes appropriately applied to the subject is indicative of the ejection fraction.

It should be understood that the expression “a diagonal pair of electrodes” relates to electrodes applied to patient's arm and leg (according to existing standards these are electrodes applied to the right arm and left leg); and the expression “a planar pair of electrodes” relates to electrodes applied to either two arms or two legs (according to existing standards these are electrodes applied to two arms).

Generally, such two pairs of electrodes, diagonal pair of electrodes and planar pair of electrodes, can be obtained using three electrodes: two applied to the patient's arm and one to his/her leg; or two electrodes applied to the legs and one to the arm.

The present invention may be used with standard ECG equipment including 6 leads interconnecting 4 electrodes. For the purposes of the present invention, signals measured from any two leads can be used.

Thus, a first data portion, indicative of a first time profile of an electrical signal measured on a planar pair of electrodes applied to a subject during a measurement session, and a second data portion, indicative a second time profile of an electrical signal measured on a diagonal pair of electrodes applied to said subject during the same measurement session, are processed. The processing comprises determining a relation between the first and second data portions, e.g. comparing the first and second data portions and upon identifying a time shift between a certain event in the first and second time profiles, generating output data indicative of the ejection fraction for said subject.

The event to be identified is typically a peak R of the QRS complex.

A time delay of the appearance of such peak R in the second time profile (diagonal pair of electrodes—Lead II in FIGS. 1A-1B) as compared to that of the first time profile (planar pair of electrodes—Lead I in FIGS. 1A-1B) is indicative of a reduction in the ejection fraction, and thus indicative of a disorder in the heart condition. When a difference between the two measured data portions exceeds a certain threshold, e.g. is higher than 10-15 msec, then the existence of abnormality on the heart condition is considered.

In some embodiments of the invention, a combination of ECG measured data and acoustic measurements is used, namely substantially simultaneously obtained data corresponding to measurements by the ECG assembly and acoustic data corresponding to measurements by an array of transducers (acoustic receivers) from a planar region of the chest or back skin surface of the patient overlying a heart chamber. The processed ECG and acoustic measured data is used for determination of ejection fraction, based on the determination of the cardiac cycle events (E1, E2, E3, E4). The use of acoustic data for determining the ejection fraction is disclosed in PCT/IL2007/001533, assigned to the assignee of the present application, which application is incorporated herein by reference.

In an embodiment of the present invention, the acoustic data is indicative of acoustic signals recorded by an array of transducers during the measurement session. It should be understood that according to the invention the acoustic data is indicative of acoustic signal(s) recorded by one or more transducers (e.g. microphones) during the measurement session, and may comprise the time profile of an electrical output of one or more transducers, and/or a certain functional of an electrical output of one or more transducers.

According to another broad aspect of the invention, there is provided a method for use in monitoring a subject's cardiac synchrony. The method comprises processing first and second measured data portions corresponding to respectively an ECG measurement and an acoustic measurement, both obtained from a subject during the same measurement session of a duration including at least two cardiovascular cycles. The processing comprises identifying certain first and second events in each of these at least two cycles in the first and second measured data, and determining a relation between them for each cycle. Then, a degree of fluctuation of this relation in the at least two cycles is determined being indicative of cardiac synchrony for said subject.

In a preferred embodiment of the invention, the two events to be identified include an initial point of the QRS complex in the first measured data and a first event (E1) in the second measured data. The first event (E1) in the acoustic measured data is a first acoustic signal peak occurring after the initial points of the QRS complex during the measurement session. The relation to be identified is a time delay between such two events, e.g. between the initial points of the QRS complex and the corresponding first acoustic signal peak (E1). When the fluctuation is well identifiable, i.e. a time delay value fluctuates from cycle to cycle, the existence of abnormality in the heart condition is considered.

Preferably, the method comprises selecting one or more acoustic signals from one or more locations, respectively, with respect to a region of interest in the subject's body. Data indicative of the so-selected acoustic signal(s) is included in the second measured data for further processing. The selected acoustic signals may be those corresponding to the output of transducers located at a certain distance from a location on the body aligned with the heart region.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1A is a schematic illustration of a monitoring system according to an embodiment of the invention;

FIG. 1B shows more specifically leads defining diagonal and planar pairs of electrodes suitable for measurements according to the present invention;

FIGS. 2A and 2B illustrate an QRS-parameter derived from an ECG signal;

FIGS. 3A and 3B compare the measurement technique of the present invention (FIG. 3B) to the conventional QRS-based technique (FIG. 3A) for a normal condition of the ejection fraction;

FIGS. 3C and 3D compare the measurement technique of the present invention (FIG. 3C) to the conventional QRS-based technique (FIG. 3C) for an abnormal condition of the ejection fraction;

FIG. 4A illustrates a correlation between the ejection fraction measurements obtainable by the conventional Echo-based and QRS-based measurements;

FIG. 4B illustrates a correlation between the ejection fraction measurements obtainable by the conventional Echo-based measurements and the technique of the present invention;

FIG. 5 is a schematic illustration of a monitoring system according to another embodiment of the invention;

FIG. 6A shows the operation principles of the monitoring system of FIG. 5 for carrying our ECG and acoustic measurements;

FIG. 6B shows the results of the QRS-based measurements applied on different electrode pairs;

FIGS. 7A and 7B show the experimental results of using the technique of the present invention for determination of the cardiac synchrony/desynchrony condition;

FIG. 8 illustrates the envelope of acoustic data measured by microphones in a 6×6 matrix of microphones;

FIGS. 9A and 9B show the results of recording and mapping a relation between the ECG and acoustic data using a matrix of microphones; and

FIGS. 10A and 10B illustrate a correlation between the measurement technique of the present invention and the Echo-based measurements, for two different groups of patients, respectively.

### DETAILED DESCRIPTION OF EMBODIMENTS

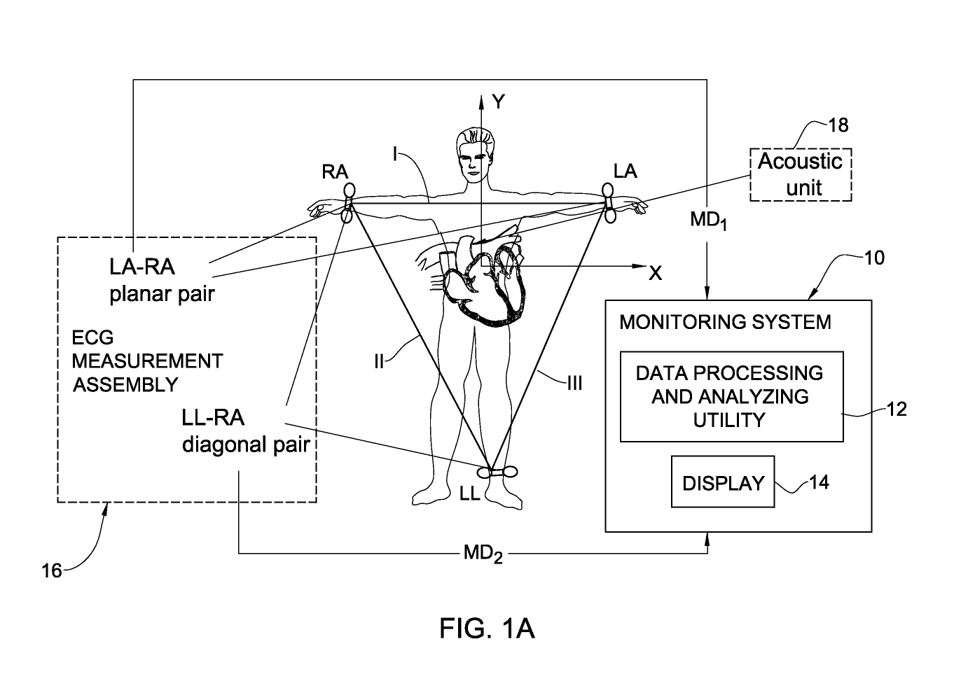

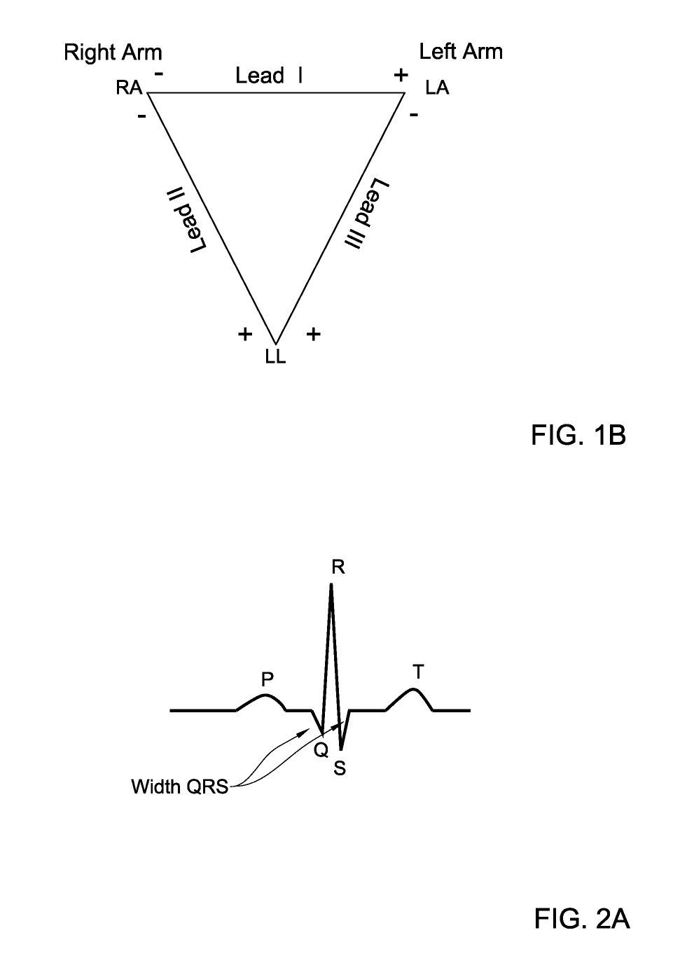

Reference is made to FIG. 1A, illustrating schematically a monitoring system, generally designated 10, configured and operable according to the present invention. The system comprises a computer system including inter alia a data processing and analyzing utility 12 and a data presentation utility 14 (e.g. display). The computer system is configured for processing measured data obtained by a conventional ECG measurement assembly 16. The computer system may and may not be directly connectable to the ECG measurement assembly.

In the present example, the ECG assembly 16 includes the standard ECG equipment including 6 leads (I, II, III, aVR, aVL and aVF) connecting four electrodes LA, RA, LL and RL (by reference or NULL). Thus, the lead I connects electrodes LA-RA, lead II-electrodes LL-RA, lead III-electrodes LL-LA, and so on. Generally, the ECG assembly includes an electrodes' arrangement formed by at least three electrodes LA, RA, LL involved in the measurements and a reference electrode RL (not shown), and includes a voltage supply/read unit (not shown). It should be noted that typically each electrode is formed by an electrode pair for applying and reading the voltages, respectively. The construction and operation of an ECG measurement unit are known per se and need not be described in details, except to note that for the purposes of the present invention the ECG unit, which collects measured data to be processed by the monitoring system, utilizes a planar pair of electrodes LA-RA and a diagonal pair of electrodes LL-RA.

The monitoring system 10 receives and processes measured data, where the latter includes a first data portion MD₁ indicative of a first time profile of an electrical signal measured on planar pair of electrodes LA-RA and a second data portion MD₂ indicative of a second time profile of an electrical signal measured on a diagonal pair of electrodes LL-RA. The measurements by the first and second pairs of electrodes LA-RA and LL-RA are applied to the patient during the same measurement session. The processing of the so-obtained measured data portions MD₁ and MD₂ includes comparing these first and second data portions to determine whether there exists a time shift between a certain event (e.g. signal peak) appearing in the first and second time profiles. Upon identifying the time shift, the system generates and displays output data indicative of the ejection fraction condition for the patient.

As shown more specifically in FIG. 1B, measurements are taken from leads I, II and III between respectively the electrodes' locations LA-RA, LL-RA, and LL-LA, respectively.

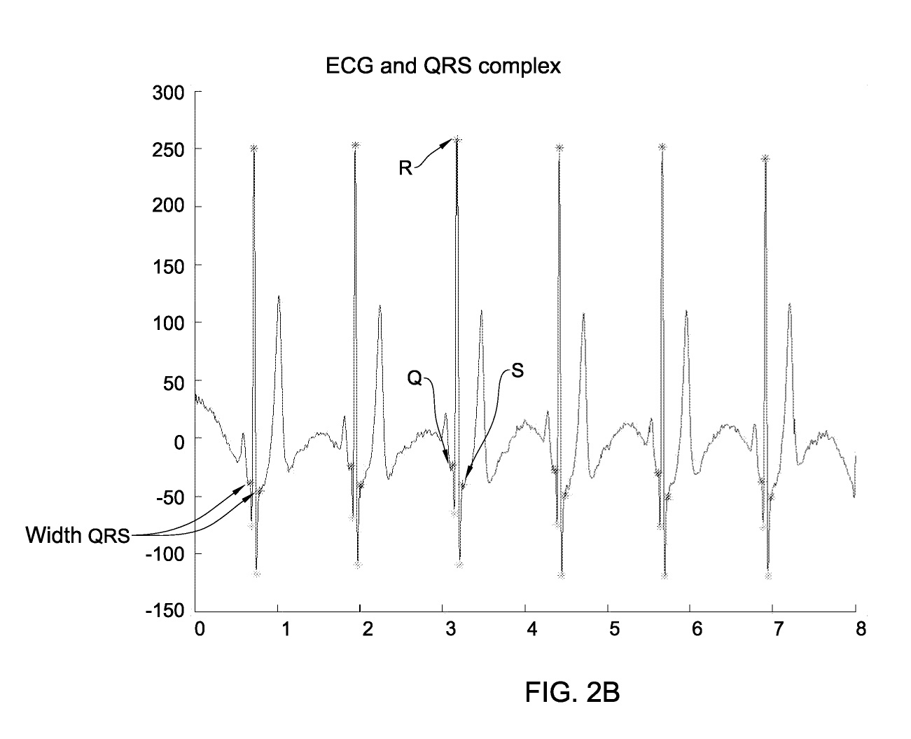

FIGS. 2A and 2B illustrate the general principles of ejection fraction estimation based on an QRS-parameter. FIG. 2B illustrates a typical ECG tracing of a normal heartbeat (or cardiac cycle) which includes the so-called QRS complex including Q-, R-, and S-waves. The QRS complex is more specifically shown in FIG. 2A.

Generally, such parameters as duration, amplitude, and morphology of the QRS complex are useful in diagnosing of cardiac arrhythmias, conduction abnormalities, ventricular hypertrophy, myocardial infarction, electrolyte derangements, and other disease states. Considering the ejection fraction determination, a condition of the ejection fraction reduction can be detected via ECG-QRS: an increase in the QRS-width corresponds to a reduction of the ejection fraction. Such ECG-QRS based measurements are insensitive to the selection of electrode-pair. In other words, all the electrode-pairs show the same results with regard to the QRS-width.

The inventors have found that although the QRS-width remains unchanged when measured on planar or diagonal electrode pairs, the QRS complexes measured by planar and diagonal electrode pairs are shifted in time. Moreover, this shift in the QRS complex at different electrode pairs is more sensitive to a change in the ejection fraction condition, than a change in the QRS-width.

Turning back to FIG. 1A, it is shown that the monitoring system 10 of the present invention may additionally be configured to process acoustic measured data. Such acoustic measured data can be obtained by using an acoustic unit 18 comprising an array of microphones (preferably a two-dimensional array) placed onto a planar region of the chest or back skin surface of the patient overlying a heart chamber whose ejection fraction is to be determined. The technique of deriving the ejection fraction condition from acoustic measurements is disclosed in PCT/IL2007/001533, assigned to the assignee of the present application, which is incorporated herein by reference.

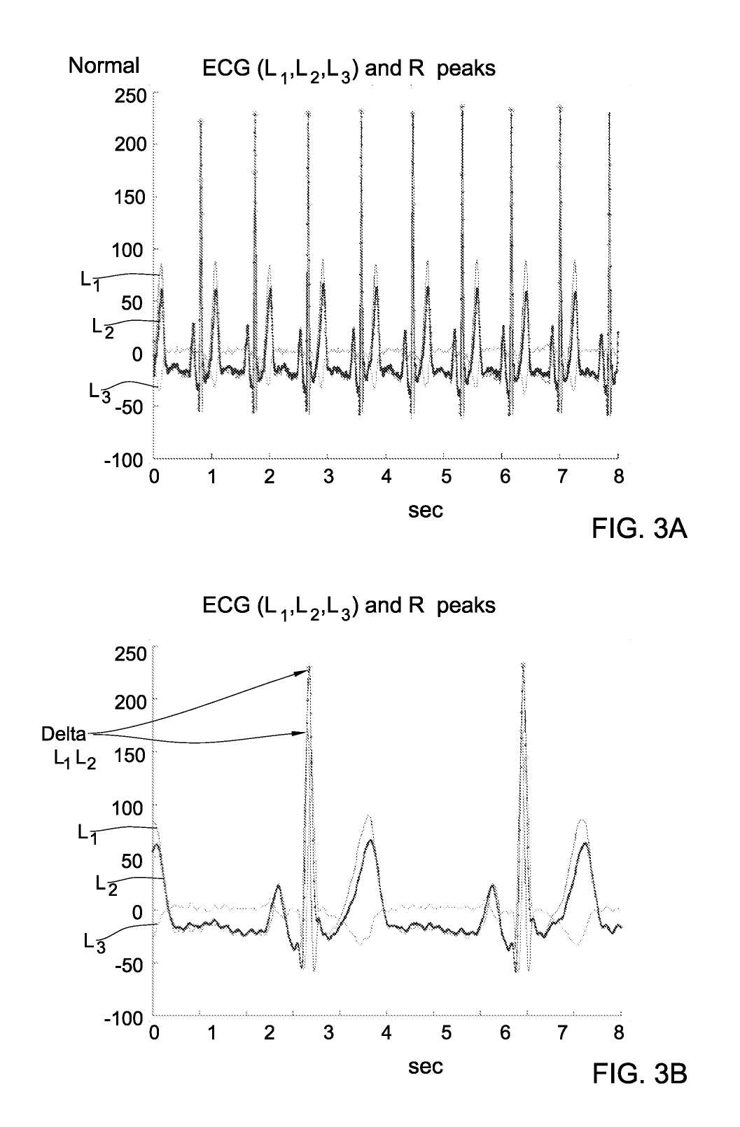

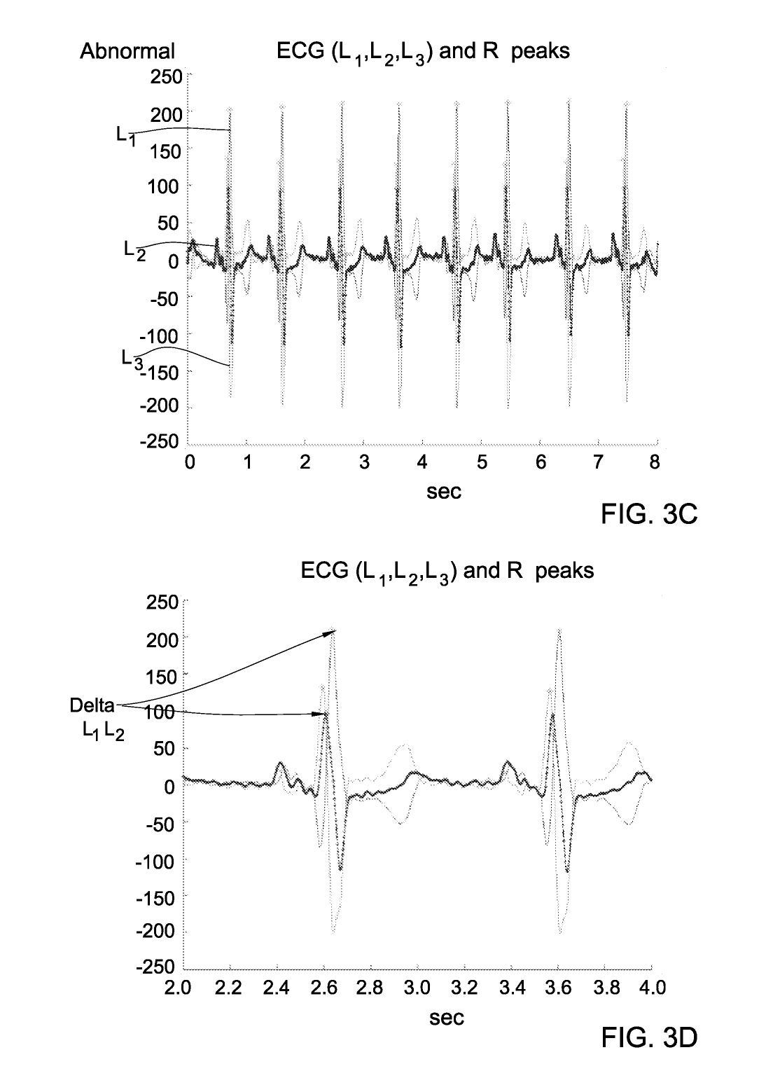

Reference is made to FIGS. 3A-3D showing experimental results for use of the method of the present invention for the determination of ejection fraction as compared to the conventional technique.

FIGS. 3A and 3B show respectively the results of the conventional QRS-based measurements and those according to the technique of the present invention, respectively, for a patient with a normal ejection fraction condition. Each figure shows three graphs L₁, L₂, and L₃ corresponding to measurement taken on the respective leads (I, II and III in FIGS. 1A and 1B), and shows R-peaks measured on all the leads. As clearly seen in FIG. 3A, the QRS-width is the same for all the measurements (a time delay exists neither between initial points of the QRS complexes at different time profiles nor between the end points of the QRS complexes), while as shown in FIG. 3B, the QRS complexes in the planar electrode pair and diagonal electrode pair measurements are slightly shifted in time by a deltaL₁L₂ time interval of about ±10 msec.

FIGS. 3C and 3D show the respective graphs for a patient with abnormal ejection fraction condition. As shown, the QRS-width still remains unchanged for measurements on different pairs of electrodes (FIG. 3C), while the well defined QRS-complex shift is clearly observed for planar and diagonal electrode pairs (FIG. 3D). The inventors have shown that the abnormal condition is characterized by such shift of up to +40 msec and higher.

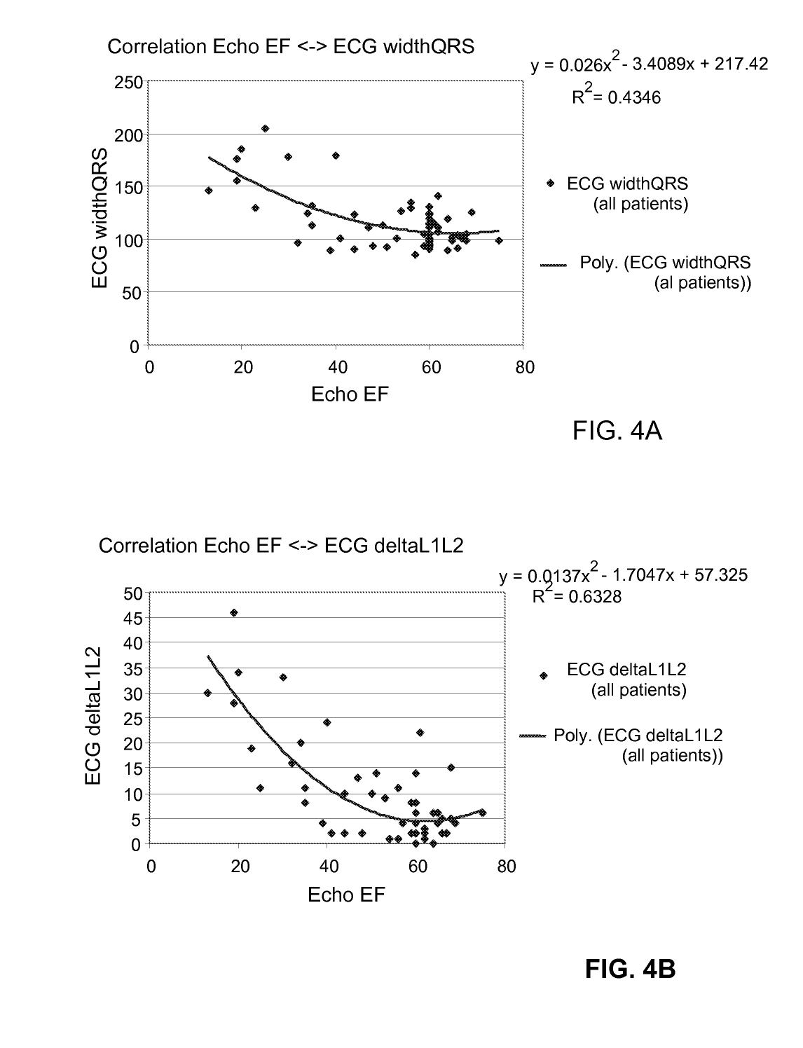

Referring to FIGS. 4A and 4B show the results of measurements taken on a group of patients (64 patients), including those with normal and abnormal ejection fraction condition. FIG. 4A shows a correlation between the ejection fraction measurements by exact calculation using Echo-based measurements and the ejection fraction determination by ECG-QRS based measurements; and FIG. 4B shows a correlation between the Echo-based measurements and the deltaL1L2-based measurements according to the present invention.

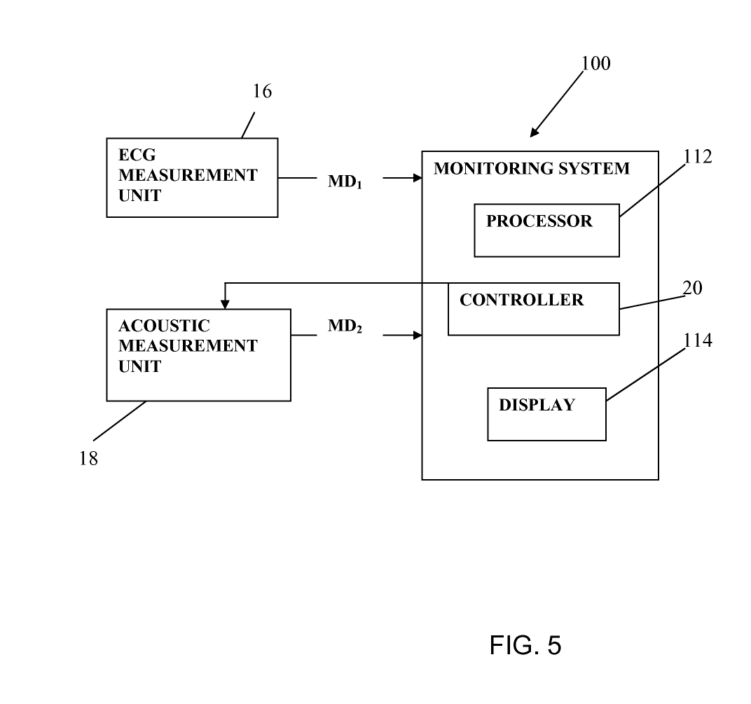

The present invention, according to yet another aspect relates to a method and system for monitoring a condition of a subject's heart synchrony/asynchrony, by determining the so-called “Vibration Systolic Desynchrony Index”. According to this technique, as shown in FIG. 5 by a way of block diagram, a monitoring system, generally designated 100, comprises as a computer system, including inter alia a processor 112 and a data presentation utility 114 (e.g. display), and is configured for processing data indicative of ECG and acoustic measurements. These measurements are carried by, respectively, a conventional ECG measurement unit 16, and an acoustic measurement unit 18. The ECG unit 16 may utilize a single or multiple pairs of electrodes. The acoustic unit 18 includes one or more transducers (e.g. microphones), preferably a matrix (two-dimensional array) of such acoustic receivers. First and second measured data portions MD₁ and MD₂, produced by the ECG and acoustic measurement units 16 and 18, respectively, are input to the monitoring system 100. These measure data portions are recorded during the same measurement session, the duration of which is such as to cover several (generally at least two) cardiovascular cycles. Monitoring system 100 preferably also includes a controller 20 associated with acoustic unit 18 for selecting acoustic receivers (microphones) to be operated during the measurement session or the measured signals of which are to be included in the acoustic measured data portion for further processing.

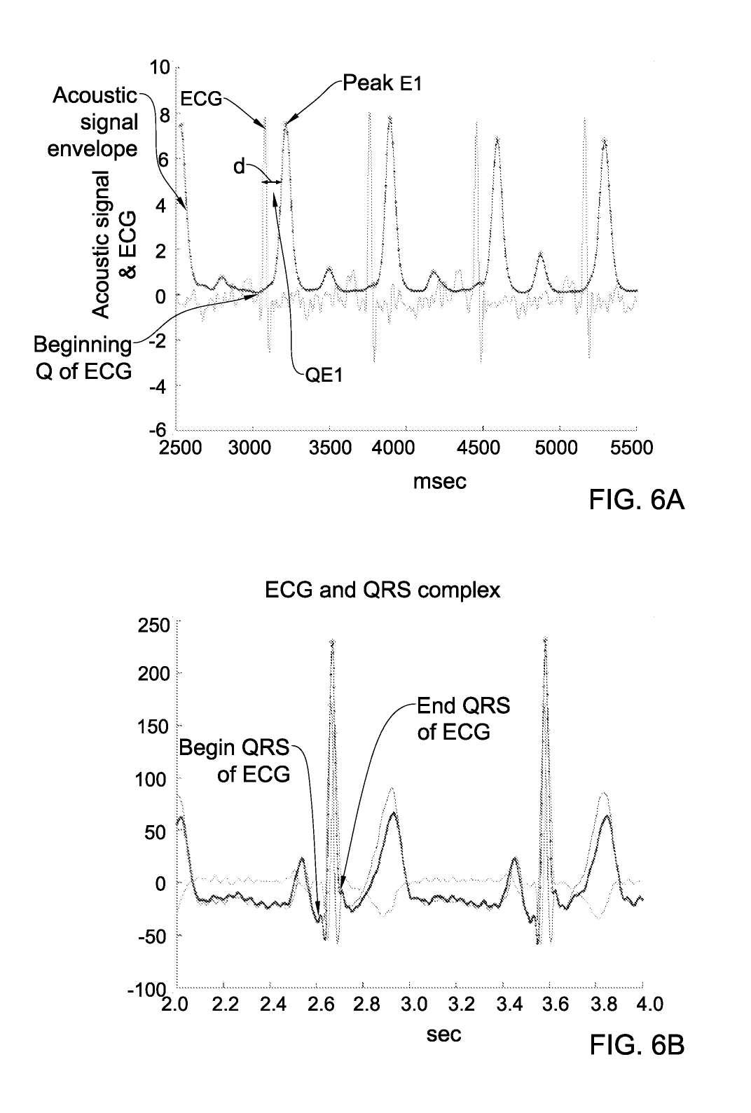

Referring to FIGS. 6A and 6B, there are illustrated experimental results for the technique of the present invention. FIG. 6A shows measured data received by the monitoring system, and including the first data portion in the form of the ECG signal and the second measured data in the form of the acoustic signal envelope. FIG. 6B shows more specifically the ECG signals measured at different electrode pairs, each characterized by the QRS-complex having its start (begin) and end points.

The inventors have found that the ECG measured data and the acoustic measured data are characterized by certain first and second events appearing in each cardiovascular cycle. In the normal cardiac synchrony condition, a relation between these is maintained for all the cardiovascular cycles, while a condition of desynchrony can be identified by a certain degree of fluctuation of this relation in the cardiovascular cycles.

Turning back to FIG. 6A, the measured data illustrated therein corresponds to a normal heart synchrony condition. The acoustic signal envelope has well defined peaks E1 and E2. This is associated with the following: One heart sound, known as “S1”, is caused by turbulence, which is in turn caused by the closure of the mitral and tricuspid valves and aortic valve opening at the start of systole between the times of the S and T points of the ECG signal. A second heart sound, known as “S2” is caused by the closure of the aortic and pulmonary valves at the end of systole after the T wave. Two events of the cardiovascular cycle, referred to herein as “E1” and “E2” are observed in the sound signal. The sound event E1 occurs at about the same time as the sound event S1 observed in the chest recording, and the sound event E2 occurs at about the same time as the sound event S1 observed in the chest recording.

As shown in FIG. 6A, a distance d between the starting points Q of the ECG and E1 peak of the acoustic signal peaks is substantially the same in all the cardiovascular cycles. This distance constitutes a relation between the events (e.g. signal peaks) in cardiovascular cycle represented in the ECG and acoustic data portions.

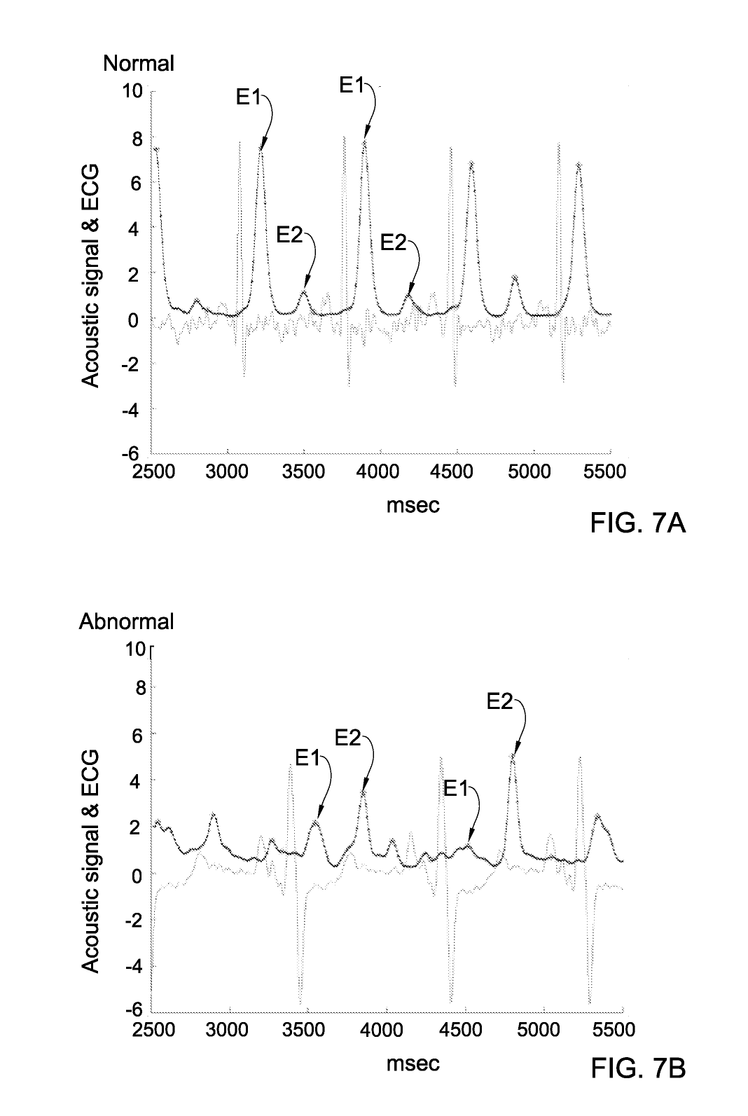

Reference is made to FIGS. 7A and 7B showing the measured data (ECG and envelope of the acoustic signals) for respectively normal and abnormal conditions of the cardiac synchrony. As clearly seen in the figures, the normal condition (FIG. 7A) is characterized by substantially the same distance between the QRS-complex and the first acoustic signal peak E1 appearing in the same cardiovascular cycle after said QRS-complex. In other words, the events characterizing a relation between the ECG and acoustic data are two successive QRS and E1 peaks. In the normal condition, the relation (distance) between these peaks is maintained for all the cycles. While in the abnormal condition (FIG. 7B), a relation between the QRS and respective E1 peak changes from cycle to cycle.

It should be noted that the acoustic data included in the respective measured data potion and further processed may be data obtained from the single microphone or from the matrix of microphones. In the latter case, this may be the entire matrix applied to the patient, or a group (sub-matrix) of the entire matrix. It should also be noted that such acoustic data may include the output of the microphone(s) itself, or a certain functional of this output signal (e.g., standard deviations for all the microphones).

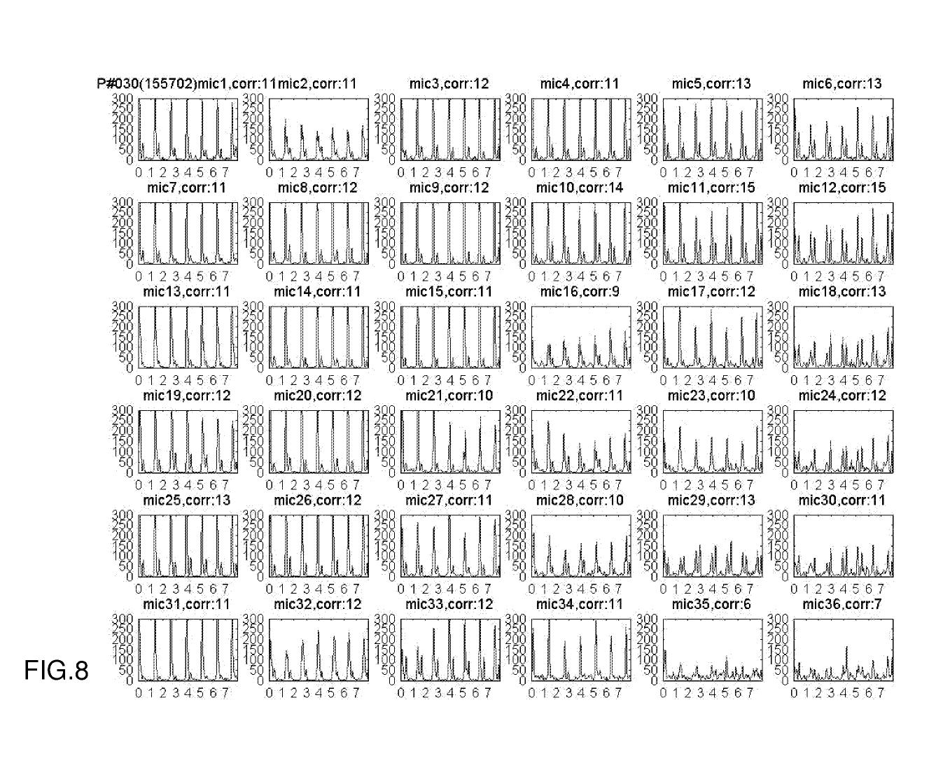

Referring to FIG. 8, there are illustrated acoustic signals (envelopes) for each of the microphones in a 6×6 matrix of microphones.

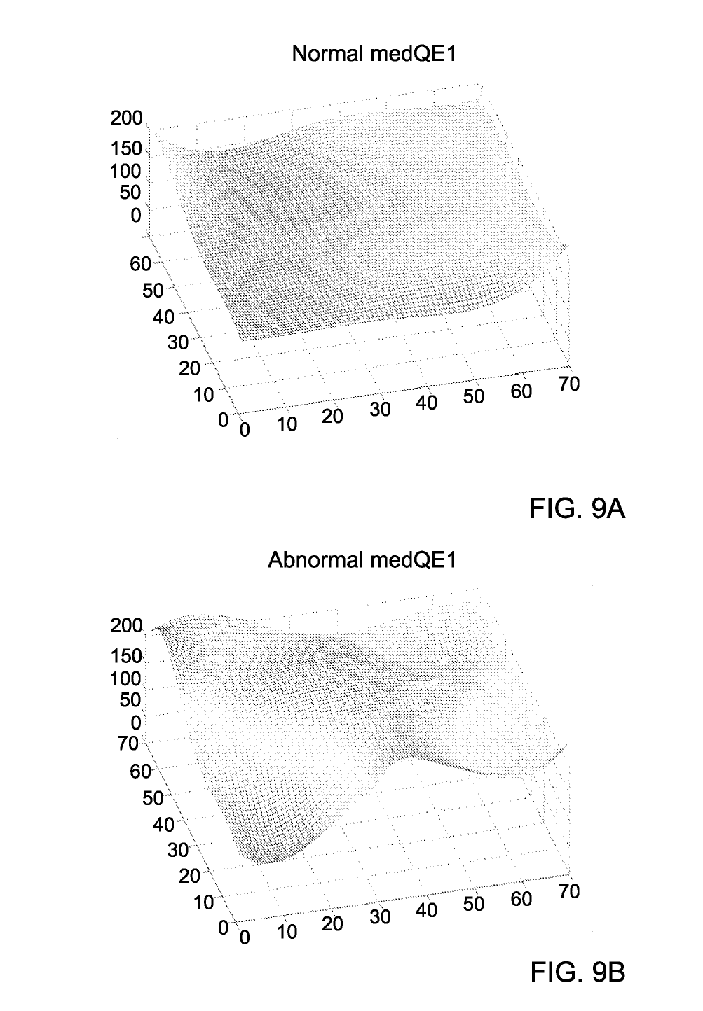

FIGS. 9A and 9B show the results of the recording and mapping of cardiac mechanical vibrations according to the invention and show the relation QE1 (i.e. distance between QRS and E1), enabling evaluation of a new vibration systolic dysynchrony index (VSDI).

The inventors have found that acoustic data associated with microphones located far away from the heart-region is more sensitive with regard to fluctuations in said relation between the events in ECG- and acoustic-signals caused by abnormality on the cardiac synchrony. In this connection, turning back to FIG. 5, the monitoring system 100 preferably includes controller 20 configured for selecting appropriate acoustic data. Controller 20 may be associated with the acoustic unit or with a memory utility of the monitoring system where measured acoustic data is stored, and may thus operate to selected acoustic receivers to be operated during the measurement session or to select the measured signals which are to be included in the acoustic measured data portion for further processing.

The following is an example of processing the output of the microphones to create the acoustic measured data:

For each microphone, a distance between the beginning of QRS complex (ECG) and E1 peak cardiac cycle event is calculated (presenting an acoustic signal envelope), for all cardiac cycles of the recording, as follows:

D_{i,j}=B_{i,j}^{QRS}−E1_{i,j}

Here, i is the microphone number which varies from 1 to N; N is the number of the microphones, and j is the cycle number which varies form 1 to n, where n is the number of cycles in a record.

For each microphone, median of the distances is calculated for all cardiac cycles of the recording, as follows:

Dᵢ^{m}=median(D_{i,j=1:n})

For each microphone, normalize standard deviations is calculated as follows:

$${σ}_{i}^{n} = {( \frac{1}{N} {\sum }_{i = 1}^{N} {( {D}_{i , j} - {D}_{i}^{m} )}^{2} )}^{\frac{1}{2}}$$

For all microphones, median of the standard deviations of the all microphones is calculated, as follows:

σ^{m}=median(σᵢⁿ)

Thirty four subjects were examined including 24 healthy controls and 40 subjects from cardiology clinics. A matrix of 5×5 transducers was applied to the chest, and the vibrations were mapped and recorded digitally. For each transducer, the interval between the onset of Q-wave and the peak of the amplitude vibration (E1 peak) was measured. VSDI for each subject was determined as the standard deviation of the difference between the median and each transducer interval. VSDI for normal controls (12.6±6.2 msec) was found to be lower than that for subjects with right ventricular pacing (30.3±9.9 msec), p<0.05. Variation of VSDI was observed in subjects with wider QRS implied various degrees of desynchrony. Thus, a simple new vibration systolic desynchrony index can differentiate between various degrees of cardiac desynchrony.

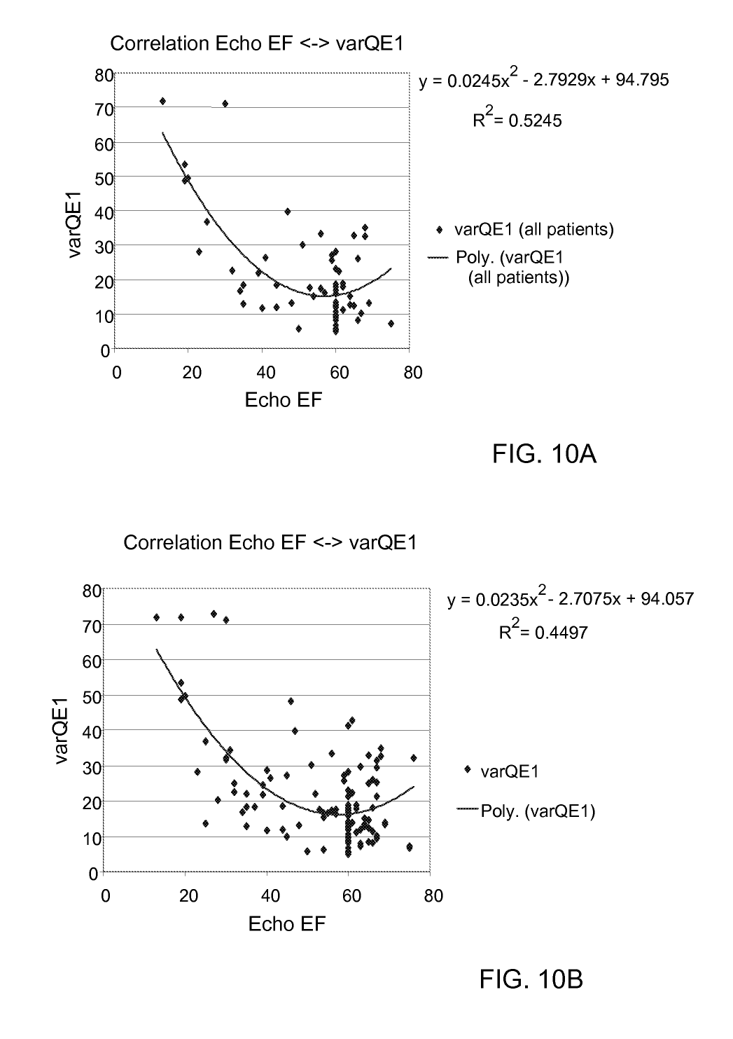

FIGS. 10A and 10B show the results of applying the technique of the present invention for determination of the cardiac synchrony to a group of subjects. As shown, the QE1-based measurements of the present invention are highly correlated with the conventional Echo-base measurements, for a 64-patients' group (FIG. 10A) and a 111-patient's group (FIG. 10B).

Thus, the present invention provides novel techniques for monitoring various parameters characterizing the heart condition. The ejection fraction parameter can be determined from a relation (time shift) between the respective signal peaks (QRS-complexes) in the ECG signals measured on planar and diagonal electrode pairs. The cardiac synchrony parameter can be determined from a behavior (degree of fluctuation) of a relation between corresponding events (e.g. QRS and E1) in the ECG and acoustic data during at least two cardiovascular cycles.

1. A method for use in monitoring a subject's ejection fraction, the method comprising: processing ECG measured data which comprises a first measured data portion indicative of a first time profile of an electrical signal measured on a planar pair of electrodes applied on a patient during a measurement session and a second measured data portion indicative a second time profile of an electrical signal measured on a diagonal pair of electrodes applied to said patient during said measurement session, said processing comprising comparing the first and second data portions and upon identifying a time shift between a predetermined event in the first and second time profiles, generating output data indicative of the ejection fraction for said subject.

2. A method according to claim 1, wherein said event is a signal peak.

3. A method according to claim 1, wherein a time delay of appearance of said event in the second time profile as compared to that of the first time profile is indicative of reduction in the ejection fraction.

4. A method according to claim 1, wherein said processing comprises processing third measured data indicative of acoustic signals recorded by an array of acoustic receivers from a planar region of the chest or back skin surface of the subject overlying a heart chamber whose ejection fraction is to be determined, and generating output data indicative of the ejection fraction of said heart chamber.

5. A monitoring system for monitoring a subject's ejection fraction, the system comprising: a data processing and analyzing utility configured for processing ECG measured data which comprises a first measured data portion indicative of a first time profile of an electrical signal measured on a planar pair of electrodes applied to a patient during a measurement session and a second measured data portion indicative a second time profile of an electrical signal measured on a diagonal pair of electrodes applied to said patient during said measurement session, said processing comprising comparing the first and second measured data portions and upon identifying a time shift between a certain event in the first and second time profiles, generating output data indicative of the ejection fraction for said patient.

6. A system according to claim 5, comprising a measurement unit comprising an electrodes arrangement configured and operable for carrying out the ECG measurement session.

7. A system according to claim 5, wherein said data processing and analyzing utility is configured for processing third measured data indicative of acoustic signals recorded by an array of acoustic receivers from a planar region of the chest or back skin surface of the subject overlying a heart chamber whose ejection fraction is to be determined, and generating output data indicative of the ejection fraction of said heart chamber.

8. A method for use in monitoring a patient's heart condition, the method comprising: processing first and second measured data portions corresponding to respectively an ECG measurement and an acoustic measurement both obtained from a patient during the same measurement session of a duration including at least two cardiovascular cycles, said processing comprising identifying predetermined first and second events in each of said at least two cycles in the first and second measured data portions, respectively, determining a relation between the first and second events for each of the at least two cycles, determining a degree of fluctuation of said relation in the at least two cycles, and generating output data indicative of cardiac synchrony for said patient.

9. A method according to claim 8, wherein said first and second events in the ECG and acoustic data portions correspond to, respectively, a QRS peak and a corresponding acoustic signal peak.

10. A method according to claim 9, wherein said corresponding acoustic signal peak is a first acoustic signal peak occurring after the QRS peak during said measurement session.

11. A method according to claim 9, wherein said relation is a time delay between the QRS and the corresponding acoustic signal peaks.

12. A method according to claim 8, wherein the second acoustic data is indicative of an acoustic signal recorded by one or more acoustic receivers during the measurement session.

13. A method according to claim 12, wherein the second measured data comprises the time profile of an electrical output of one or more acoustic receivers.

14. A method according to claim 12, wherein the second measured data comprises a certain functional of an electrical output of one or more acoustic receivers.

15. A method according to claim 8, wherein the second acoustic data is indicative of acoustic signals recorded by an array of acoustic receivers during the measurement session.

16. A method according to claim 15, comprising selecting one or more acoustic signals from one or more locations, respectively, with respect to a region of interest in the patient's body to include data indicative thereof in said second measured data for said processing.

17. A method according to claim 16, wherein said one or more acoustic signals correspond to the output of one or more acoustic receivers, respectively, located at a certain distance from a location on the body aligned with the heart region.

18. A system for use in monitoring a patient's heart condition, the system comprising: a data processing and analyzing utility, configured and operable for receiving a first measured data portion indicative of an ECG measurement and a second measured data portions indicative of an acoustic measurement, both obtained from a patient during the same measurement session of a duration including at least two operative cycles, and configured and operable for processing said measured data portions for identifying certain first and second events in each of said at least two cycles in the first and second measured data portions, respectively, determining a relation between the first and second events for each of the at least two cycles, determining a degree of fluctuation of said relation in the at least two cycles, and generating output data indicative of cardiac synchrony for said patient.

19. A system according to claim 18, comprising an ECG measurement unit and an acoustic measurement unit connectable to said data processing and analyzing utility.

20. A system according to claim 18, comprising a controller adapted for selectively operating one or more acoustic receivers of said acoustic unit to carry out said acoustic measurement.

21. A system according to claim 18, comprising a controller utility adapted for selecting one or more acoustic signals from one or more locations, respectively, with respect to a region of interest in the patient's body to transmit data indicative thereof to said data processing and analyzing utility to be processed as said second measured data.
